(19) United States
(12) Patent Application Publication
(10) Pub. No.: US 2026/0015302 A1
(43) Pub. Date: Jan. 15, 2026

(54) METHOD FOR PREPARING CYCLOALKENE FROM DOUBLE BOND-CONTAINING POLYMER AND METHOD FOR RECYCLING WASTE RUBBER USING THE SAME

(71) Applicant: Korea Advanced Institute of Science and Technology, Daejeon (KR)

(72) Inventors: Soon Hyeok HONG, Daejeon (KR); Kyungmin CHOI, Daejeon (KR); Beomsoon PARK, Daejeon (KR); Kyoungil CHO, Daejeon (KR)

(73) Assignee: Korea Advanced Institute of Science and Technology, Daejeon (KR)

(21) Appl. No.: 19/222,016

(22) Filed: May 29, 2025

(30) Foreign Application Priority Data
May 29, 2024 (KR) 10-2024-0069835

(51) Int. Cl. C07C 4/22; C07C 6/04; C08J 11/28

(52) U.S. Cl. CPC C07C 4/22; C07C 6/04; C08J 11/28; C07C 2531/22; C07C 2531/24; C08J 2309/00

(57) ABSTRACT

Provided are a method for preparing cycloalkene including: depolymerizing a double bond-containing polymer in the presence of an isomerization catalyst and a metathesis catalyst having a N-heterocyclic carbene ligand to prepare (C5-C7)cycloalkene, and a method for recycling waste rubber using the same.

### CROSS-REFERENCE TO RELATED APPLICATIONS

This application claims the benefit of priority under 35 U.S.C. § 119 to Korean Patent Application No. 10-2024-0069835, filed May 29, 2024, the content of which is incorporated herein by reference in its entirety.

### TECHNICAL FIELD

The following disclosure relates to a method for preparing cycloalkene from a double bond-containing polymer, and a method for chemically recycling waste rubber using the same.

### BACKGROUND

125 thousand tons of rubber are synthesized every year and are widely used as disposable materials, in the manufacture of tires, and the like. Despite the essential properties and wide range of application fields of the rubber, treatment of used waste rubber and waste tire still relies on mechanical recycling or use as fuel. Mechanical recycling has limitations in that the mechanical properties of rubber deteriorate with each recycling, restricting its use to applications with low performance requirements. Therefore, chemical recycling methods, such as thermal decomposition, hydrolysis, and ozone decomposition, have been proposed to recover olefin-based products from waste rubber.

However, the chemical recycling of waste rubber requires complicated and sophisticated technology and harsh reaction conditions and has limitations such as low selectivity and yield of the product obtained by chemical decomposition and obtaining complicated mixtures.

### RELATED ART DOCUMENTS

### Non-Patent Document

(Non-patent document 1) Catal. Sci. Technol., 2016, 6, 7708-7717

### SUMMARY

An embodiment of the present invention is directed to providing a method for preparing cycloalkene by depolymerization of a double bond-containing polymer.

Another embodiment of the present invention is directed to providing a method for chemically recycling waste rubber using the method for preparing cycloalkene.

Still another embodiment of the present invention is directed to providing a method for preparing cycloalkene from waste rubber such as waste tires.

In one general aspect, a method for preparing cycloalkene includes: depolymerizing a double bond-containing polymer in the presence of an isomerization catalyst and a metathesis catalyst having a N-heterocyclic carbene ligand to prepare (C5-C7)cycloalkene.

The double bond-containing polymer may be polyalkadiene, polycycloalkene, or a copolymer thereof.

The double bond-containing polymer may be a polymer derived from (C4-C10)alkadiene or (C5-C10)cycloalkene, or a copolymer including the same.

The double bond-containing polymer may be a rubber.

The metathesis catalyst may be a ruthenium complex including a N-heterocyclic carbene ligand.

The N-heterocyclic carbene ligand may be represented by the following Chemical Formula 1:

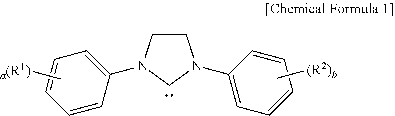

wherein R¹ and R² are independently of each other (C1-C4)alkyl, and
a and b are independently of each other an integer of 1 to 3.

The N-heterocyclic carbene ligand may be selected from the following structures:

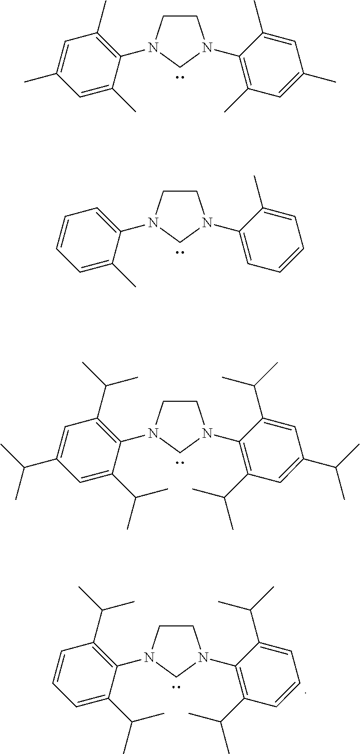

The isomerization catalyst may include a phosphine ligand and a central metal.

The isomerization catalyst may be [Pd(μ-Br)^{t}Bu₃P]₂, RuHCl(CO)(PPh₃)₃, Ru(Me-allyl)₂COD, Ru-MACHO, Pd/C, or Ru/C.

The depolymerization reaction may be performed at a temperature of 50 to 150° C.

The isomerization catalyst and the metathesis catalyst may be used at a mole ratio of 1:0.1 to 1.5.

The polyalkadiene may be selected from polybutadiene, polyisoprene, polypentadiene, and polychlorobutadiene.

The polycycloalkene may be selected from polycyclooctene, polycyclopentene, and polycyclooctadiene.

In another general aspect, a method for recycling waste rubber includes: grinding waste rubber to obtain waste rubber powder; and depolymerizing the waste rubber powder in the presence of an isomerization catalyst and a metathesis catalyst having a N-heterocyclic carbene ligand to prepare (C5-C7)cycloalkene.

Other features and aspects will be apparent from the following detailed description, the drawings, and the claims.

### BRIEF DESCRIPTION OF THE DRAWINGS

FIG. 1 schematizes a method for depolymerizing waste rubber according to an exemplary embodiment.

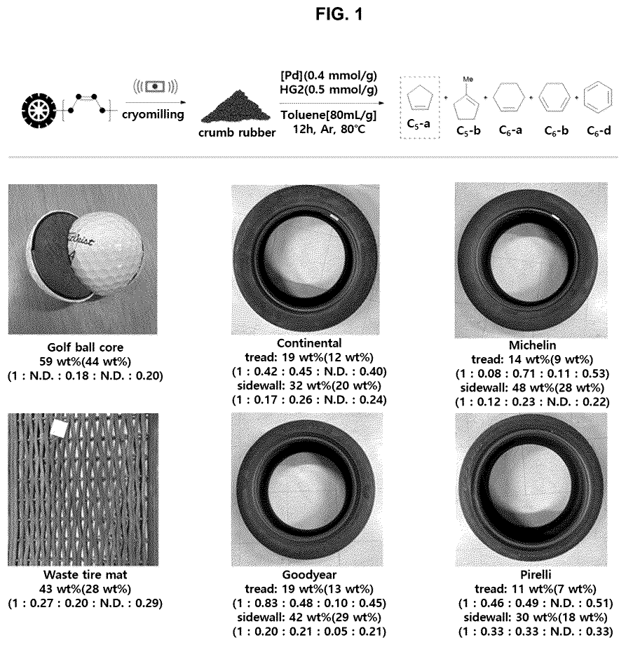

### DETAILED DESCRIPTION OF EMBODIMENTS

In the present specification, unless otherwise defined, all technical terms and scientific terms have the same meanings as those commonly understood by a person skilled in the art to which the present invention pertains. The terms used herein are only for effectively describing a certain specific example and are not intended to limit the present invention.

The singular form used in the present specification may be intended to also include a plural form, unless otherwise indicated in the context.

Throughout the present specification, unless otherwise particularly stated, the word “comprise”, “equipped”, “contain”, or “have” does not mean the exclusion of any other constituent element, but means further inclusion of other constituent elements, and elements, materials, or processes which are not further listed are not excluded.

The numerical range used in the present specification includes all values within the range including the lower limit and the upper limit, increments logically derived from the form and spanning of a defined range, all double limited values, and all possible combinations of the upper limit and the lower limit in the numerical range defined in different forms. Unless otherwise defined in the present specification, values which may be outside a numerical range due to experimental error or rounding off of a value are also included in the defined numerical range.

Unless otherwise particularly defined in the present specification, “about” may be considered as a value within 30%, 25%, 20%, 15%, 10%, or 5% of a stated value.

In the present specification, “cycloalkene” refers to an alicyclic compound having one or more carbon-carbon double bonds.

Hereinafter, the present disclosure will be described in detail. However, it is only illustrative, and the present disclosure is not limited to the specific exemplary embodiment which is illustratively described.

An exemplary embodiment of the present invention provides a method for preparing cycloalkene by depolymerization of a double bond-containing polymer and a method for recycling waste rubber using the same.

Specifically, the method for preparing cycloalkene according to an exemplary embodiment may include: depolymerizing a double bond-containing polymer in the presence of an isomerization catalyst and a metathesis catalyst having a N-heterocyclic carbene ligand to prepare (C5-C7)cycloalkene.

The method for preparing cycloalkene according to an exemplary embodiment may provide (C5-C7)cycloalkene with a high selectivity and a high yield by performing the isomerization reaction and the metathesis reaction, specifically the isomerization reaction and the ring-closing metathesis depolymerization (RCMD) reaction of the double bond-containing polymer simultaneously.

The double bond-containing polymer may include polyalkadiene, polycycloalkene, copolymers thereof, crosslinked polymer thereof, or the like.

The polyalkadiene may be a polymer derived from a diene-based compound, and the diene-based compound may be a conjugated diene-based compound or a non-conjugated diene-based compound. Specifically, the polyalkadiene may be a polymer derived from conjugated or non-conjugated (C4-C10)alkadiene, conjugated or non-conjugated (C4-C8)alkadiene, or conjugated or non-conjugated (C4-C6)alkadiene, and the diene-based compound may be butadiene, isoprene, 1,3-pentadiene, 2,3-dimethyl-1,3-butadiene, or 2-chloro-1,3-butadiene, but is not limited thereto.

The polycycloalkene may be a polymer derived from cycloalkene, and the cycloalkene may contain one or two or more double bonds, may be (C5-C12)cycloalkene, and for example, may be cyclopentene, cyclohexene, cycloheptene, or cyclooctene; cyclodiene such as cyclopentadiene, 1,2-cyclohexadiene, 1,3-cyclohexadiene, 1,2-cyclooctadiene, 1,3-cyclooctadiene, 1,4-cyclooctadiene; or cyclotriene such as 1,5,9-cycloodecatriene, but is not limited thereto.

The double bond-containing polymer may be, for example, polybutadiene, polyisoprene, polypiperylene, polycyclooctene, a butadiene-styrene copolymer, a styrene-butadiene-styrene copolymer, a butadiene-acrylonitrile copolymer, neoprene, a crosslinked rubber thereof, or the like.

The double bond-containing polymer may be rubber, the rubber may be natural rubber or synthetic rubber, and the rubber may be a pre-vulcanized rubber or vulcanized rubber product.

The (C5-C7)cycloalkene prepared from the preparation method according to an exemplary embodiment may be substituted or unsubstituted (C5-C7)cycloalkene. Specifically, the substituted (C5-C7)cycloalkene may be substituted by a specific substituent depending on the type of double bond-containing polymer, and the substitution may be substitution with one or more substituents selected from the group consisting of hydroxy, amino, cyano, nitro, carboxyl, chloro, C1-C20 alkyl, and C1-C20 alkoxy.

The metathesis catalyst may be used without limitation as long as it is an olefin metathesis catalyst capable of metathesis, but as a non-limiting example, may be a ruthenium (Ru) complex including a N-heterocyclic carbene ligand.

The N-heterocyclic carbene ligand may be represented by the following Chemical Formula 1:

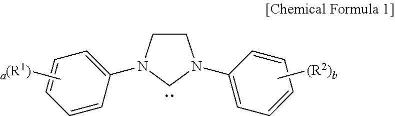

Wherein
R¹ and R² are independently of each other (C1-C4)alkyl, and
a and b are independently of each other an integer of 1 to 3.

As an example, R¹ and R² may be independently of each other methyl or isopropyl.

As an example, R¹ and R² may be identical to each other and methyl or isopropyl.

As an example, a and b may be identical to each other and 1 to 3.

As an example, the N-heterocyclic carbene ligand may be selected from the following structures:

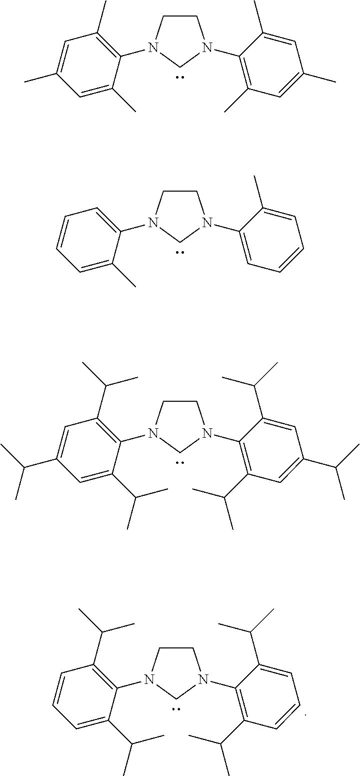

As an example, the metathesis catalyst including the N-heterocyclic carbene ligand may be selected from the following Chemical Formulae A to C:

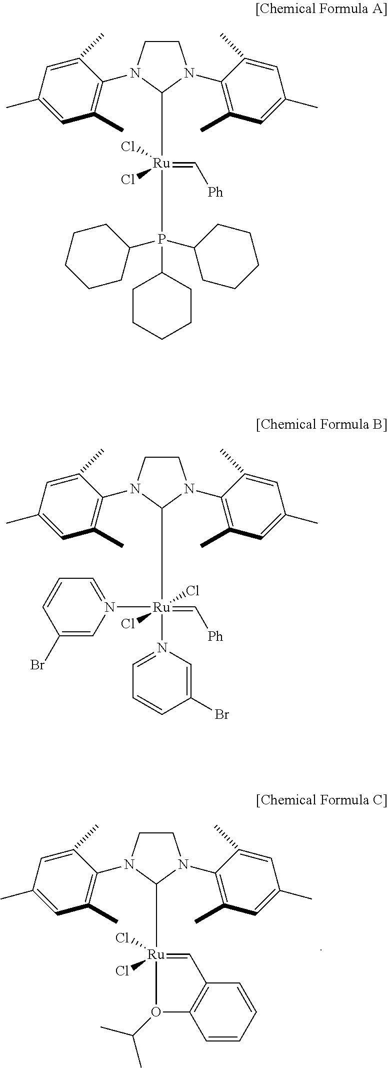

The isomerization catalyst may be used without limitation as long as it is a catalyst capable of isomerization of a double bond and may be an isomerization catalyst including a ligand and a central metal, the ligand may be a phosphine ligand such as trialkyl (C1-C7)phosphine or triaryl (C6-C12)phosphine, an allyl ligand such as 2-methyl-allyl, or a cyclodiene ligand such as 1,5-cyclooctadiene, the central metal may be ruthenium (Ru) or palladium (Pd), and the catalyst may be a heterogeneous Pd catalyst or a heterogeneous Ru catalyst. Specifically, the isomerization catalyst may include a phosphine ligand and a central metal such as ruthenium or palladium, and may produce (C5-C7)cycloalkene with a better yield.

The isomerization catalyst may be a transfer hydrogenation catalyst and may be a ruthenium (Ru) or iridium (Ir) catalyst having a ligand capable of tridentate coordination of phosphorus, nitrogen, and phosphorus.

The isomerization catalyst may be, as a non-limiting example, [Pd(μ-Br)^{t}Bu₃P]₂, RuHCl(CO)(PPh₃)₃, Ru(Me-allyl)₂COD, Ru-MACHO, Pd/C, or Ru/C.

The depolymerization reaction may be performed at a temperature of 50 to 250° C., 50 to 150° C., 50 to 100° C., or 60 to 90° C., or at a temperature between the numerical range, and is preferred since (C5-C7)cycloalkene may be prepared with a better yield.

The depolymerization reaction may be performed under an organic solvent, and the organic solvent may be an aromatic hydrocarbon solvent such as benzene, toluene, xylene, ethylbenzene, or methylnaphthalene.

The isomerization catalyst may be used at 0.001 to 1 mmol/g, 0.001 to 0.5 mmol/g, 0.001 to 0.1 mmol/g, 0.01 to 0.1 mmol/g, or 0.03 to 0.1 mmol/g, based on the weight of the double bond-containing polymer, but is not limited thereto.

The metathesis catalyst may be used at 0.0005 to 0.5 mmol/g, 0.0005 to 0.01 mmol/g, 0.001 to 0.1 mmol/g, or 0.01 to 0.1 mmol/g, based on the weight of the double bond-containing polymer, but is not limited thereto.

The isomerization catalyst and the metathesis catalyst may be used at a mole ratio of 1:0.001 to 5, 1:0.001 to 2, 1:0.001 to 1.5, 1:0.001 to 1, preferably 1:0.01 to 1, 1:0.05 to 1, or 1:0.5 to 1, but is not limited thereto.

Another exemplary embodiment of the present invention provides a method for recycling waste rubber using the method for preparing cycloalkene.

The method for recycling waste rubber according to an exemplary embodiment of the present invention may include: grinding waste rubber to obtain waste rubber powder; and depolymerizing the waste rubber powder in the presence of an isomerization catalyst and a metathesis catalyst having a N-heterocyclic carbene ligand to prepare (C5-C7)cycloalkene.

Since the double bond-containing polymer, the isomerization catalyst, and the N-heterocyclic carbene ligand are as described above, the description thereof will be omitted.

The rubber component of the waste rubber means including natural rubber, synthetic rubber, or vulcanized rubber, and an example of the waste rubber may be wastes such as nitrile gloves, golf ball core, O-ring, and tires, but is not limited thereto.

A method for grinding the waste rubber is not limited as long as rubber products are cut or made small, but may use a cutter, and an example of the cutter may be products such as nippers, grinders, cutter knives, crushers, and ball mills, but is not limited thereto. In addition, waste rubber cut into a size of 1 to 10 cm may be ground into smaller particles by a grinder or a ball mill in a low temperature condition of 0 to −78° C., but is not limited thereto.

The metathesis catalyst may be used at 0.01 to 50 mmol/g, 0.01 to 20 mmol/g, 0.01 to 10 mmol/g, or 0.01 to 1 mmol/g, based on the weight of the waste rubber powder, but is not limited thereto.

The metathesis catalyst may be used at 0.01 to 50 mmol/g, 0.01 to 20 mmol/g,

0.01 to 10 mmol/g, or 0.01 to 1 mmol/g, based on the weight of the waste rubber powder, but is not limited thereto.

Hereinafter, the exemplary embodiments described above will be described in detail through the following examples. However, the following examples are only for description, and do not limit the right scope.

The physical properties of the examples were measured as follows:

### 1. Gas Chromatography

GC-FID (Agilent 7890B equipped with flame ionization detector) was used, and as a column, Petrocol DH 50.2 (Supelco, Bellefonte, PA, length: 50 m, Diameter: 0.2 mm, film: 0.5 μm) was used. The pressure of the column was 27,497 psi, a carrier gas flow was 2.2 mL/min, and a split ratio was set to 20:1. The temperature of an oven was initially maintained at 35° C. for 10 minutes, increased to 80° C. at 5° C./min, maintained for 2 minutes, increased to 100° C. at 10° C./min, maintained for 1.5 minutes, increased to 220° C. at 20° C./min, maintained for 5.5 minutes, increased to 300° C. at 5° C./min, and maintained for 5 minutes, and analysis was performed for about 60 minutes.

The types of catalysts described in the following table are as follows:

[Pd]: [Pd(μ-Br)^{t}Bu₃P]₂

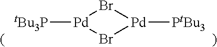

[Ru]: RuHCl(CO)(PPh₃)₃

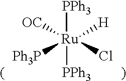

Grubbs 1^{st}: Grubbs 1^{st} generation metathesis catalyst

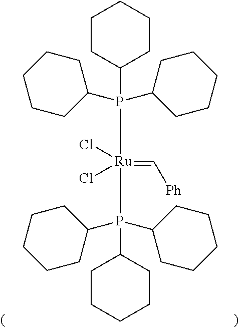

Grubbs 2^{nd}: Grubbs 2^{nd} generation metathesis catalyst

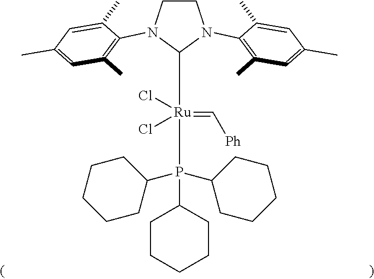

Grubbs 3^{rd}: Grubbs 3^{rd} generation metathesis catalyst

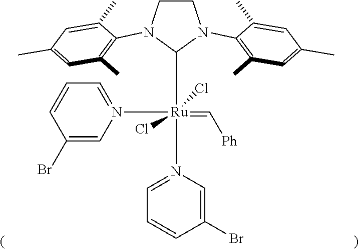

HG2 Hoveyda-Grubbs 2^{nd} generation metathesis catalyst

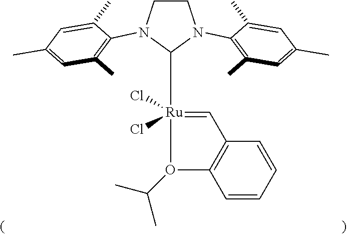

[Example 1]

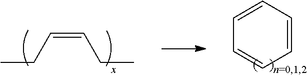

| | | | | | |
|---|---|---|---|---|---|
| 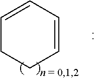 | 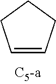 | 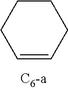 | 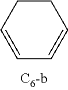 | 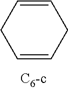 | 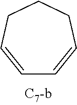 |

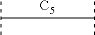

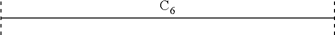

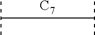

0.80 mmol of cis-polybutadiene (Aldrich, M_{w}=200-300 kD), 0.004 mmol (3.2 mg in 0.2 mL DCM) of an isomerization catalyst [Pd(μ-Br)^{t}Bu₃P]₂, and 0.001 mmol (0.6 mg in 0.1 mL DCM) of a Hoveyda-Grubbs 2^{nd} generation metathesis catalyst were added to a 10 mL Schlenk flask, and toluene was added thereto to make the solution 0.2 M. The mixed solution was stirred at 80° C. for 12 hours, 20 μL of dodecane as an internal standard was added, a part of the reaction solution was filtered through a short silica (DCM eluent) pad, and the product was analyzed by gas chromatography (GC). The yield of (C5-C7)cycloalkene and the composition of the obtained (C5-C7)cycloalkene were analyzed by GC analysis and shown in the following Table 1.

### Examples 2 to 4

The process was performed in the same manner as in Example 1, except that the type of catalyst was changed as shown in the following Table 1.

### Examples 5 and 6

The process was performed in the same manner as in Example 1, except that the reaction temperature was changed to 60° C. and 100° C.

### Comparative Example 1

The process was performed in the same manner as in Example 1, except that the isomerization catalyst was not used.

### Comparative Example 2

The process was performed in the same manner as in Example 1, except that the metathesis catalyst was not used.

### Comparative Example 3

The process was performed in the same manner as in Example 1, except that a Grubbs 1^{st} generation metathesis catalyst was used instead of the Hoveyda-Grubbs 2^{nd} generation metathesis catalyst.

TABLE 1

| | Catalyst | | | | | |
|---|---|---|---|---|---|---|
| | Isomerization | Metathesis | Yield | Ratio (%) | | |
| | catalyst | catalyst | (wt %) | C₅ | C₆ | C₇ |
| Example 1 | [Pd] | HG2 | 82 | 70 | 24 | 7 |
| Example 2 | [Ru] | HG2 | 75 | 65 | 31 | 4 |
| Example 3 | [Pd] | Grubbs | 65 | 68 | 24 | 8 |
| | | 2^{nd} | | | | |
| Example 4 | [Pd] | Grubbs | 52 | 68 | 18 | 14 |
| | | 3^{rd} | | | | |
| Example 5 | [Pd] | HG2 | 74 | 68 | 27 | 4 |
| Example 6 | [Pd] | HG2 | 65 | 68 | 18 | 14 |
| Comparative | — | HG2 | 3 | 63 | 31 | 6 |
| Example 1 | | | | | | |
| Comparative | [Pd] | — | N.D. | N.D. | N.D. | N.D. |
| Example 2 | | | | | | |
| Comparative | [Pd] | Grubbs | 3 | 68 | N.D. | 32 |
| Example 3 | | 1^{st} | | | | |

### Example 7

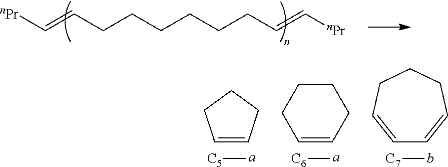

0.40 mmol of polycyclooctene (26.0 kD), 0.004 mmol (3.2 mg in 0.2 mL DCM) of the isomerization catalyst RuHCl(CO)(PPh₃)₃, and 0.001 mmol (0.6 mg in 0.1 mL DCM) of a Hoveyda-Grubbs 2^{nd} generation catalyst were added to a 10 mL Schlenk flask, and toluene was added thereto to make the solution 0.1 M. The mixed solution was stirred at 80° C. for 12 hours, 20 μL of dodecane as an internal standard was added, a part of the reaction solution was filtered through a short silica (DCM eluent) pad, analysis was performed by gas chromatography (GC), and the analysis results are shown in the following Table 2.

### Example 8

The process was performed in the same manner as in Example 7, except that the type of catalyst was changed as shown in the following Table 2.

### Comparative Example 4

The process was performed in the same manner as in Example 7, except that the isomerization catalyst was not used.

### Comparative Example 5

The process was performed in the same manner as in Example 7, except that the metathesis catalyst was not used.

TABLE 2

| | Catalyst | | | | | |
|---|---|---|---|---|---|---|
| | Isomerization | Metathesis | Yield | Ratio (%) | | |
| | catalyst | catalyst | (wt %) | C₅ | C₆ | C₇ |
| Example 7 | [Ru] | HG2 | 62 | 4 | 92 | 4 |
| Example 8 | [Pd] | HG2 | 56 | 16 | 77 | 7 |
| Comparative | — | HG2 | 31 | 4 | 26 | 69 |
| Example 4 | | | | | | |
| Comparative | [Ru] | — | N.D. | N.D. | N.D. | N.D. |
| Example 5 | | | | | | |

### Example 9

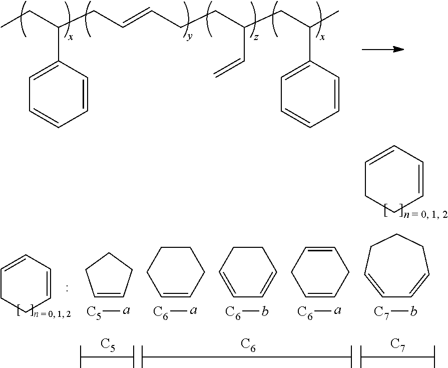

The process was performed in the same manner as in Example 1, except that polystyrene-b-polybutadiene-b-polystyrene (Aldrich 432490) was used instead of polybutadiene. As a result of GC analysis, the yield of (C5-C7)cycloalkene was 35 wt %, and it was confirmed that the composition ratio of cycloalkene was 64% of C₅ cycloalkene, 26% of C₆ cycloalkene, and 10% of C7 cycloalkene.

### Example 10

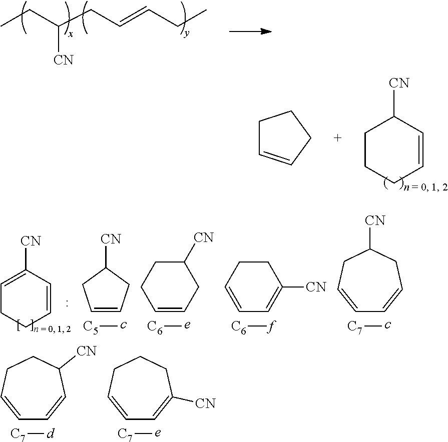

The process was performed in the same manner as in Example 1, except that poly(acrylonitrile-co-butadiene) (Aldrich 180912) was used instead of polybutadiene. As a result of gas chromatography (GC) analysis, the yield of (C5-C7)cycloalkene was 42 wt %, and it was confirmed that nitrile-substituted (C5-C7)cycloalkene was obtained by GC analysis.

### Examples 11 to 18

Waste rubber cut into small pieces with a cutter was made into particles by ball mill equipment at a low temperature to obtain waste rubber powder (crumb rubber).

50 mg of waste rubber, 0.2 mmol (0.04 mmol/g) of the isomerization catalyst [Pd(μ-Br)^{t}Bu₃P]₂, and 0.02 mmol (0.4 mmol/g) of the Hoveyda-Grubbs 2^{nd} generation metathesis catalyst were added to a 10 mL Schlenk flask, and 4 mL of toluene (80 mL/g) was added. The mixed solution was stirred at 80° C. for 12 hours, 20 μL of dodecane as an internal standard was added, a part of the reaction solution was filtered through a short silica (DCM eluent) pad, and the product was analyzed by gas chromatography (GC). It was confirmed that unsubstituted (C5-C7)cycloalkene and methyl and nitrile-substituted (C5-C7)cycloalkene were obtained by GC analysis, and the yield and cycloalkene selectivity were calculated and shown in the following Table 3.

The type of waste rubber used in Examples 11 to 18 was a nitrile glove, a golf ball core, a Buna-N O-ring, a waste tire mat, and tire (Continental, Goodyear, Michelin, and Pirelli), respectively.

TABLE 3

| | Yield | Ratio | | |
|---|---|---|---|---|
| | (wt %) | (%) | | |
| Example 11 (nitrile glove) | 50 | 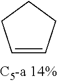 | 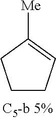 | 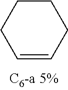 |

| | | |
|---|---|---|
| 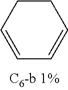 | 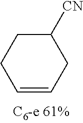 | 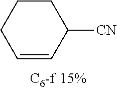 |

| Example 12 (golf ball core) | 46 | 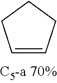 | 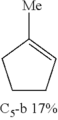 | | 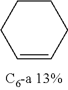 |
|---|---|---|---|---|---|
| Example 13 (Buna-N O-ring) | 33 | 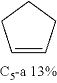 | 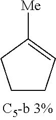 | | 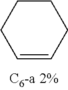 |
| | | 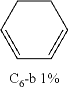 | 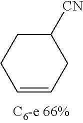 | | 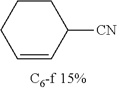 |
| Example 14 (waste tire mat) | 28 | 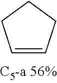 | 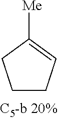 | 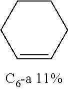 | 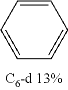 |

| | | | | | |
|---|---|---|---|---|---|
| Example 15 (Continental tire) | Sidewall: 21 | 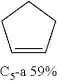 | 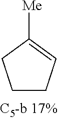 | 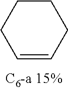 | 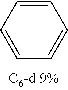 |

| | | | | |
|---|---|---|---|---|
| Tread: 13 | 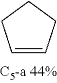 | 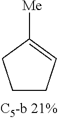 | 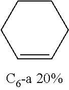 | 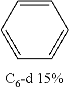 |

| | | | | | | |
|---|---|---|---|---|---|---|
| Example 16 (Goodyear tire) | Sidewall: 29 | 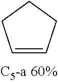 | 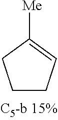 | 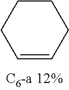 | 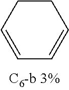 | 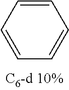 |
| | Tread: 13 | 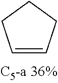 | 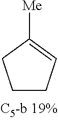 | 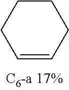 | 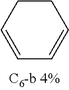 | 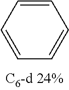 |

| | | | | | |
|---|---|---|---|---|---|
| Example 17 (Michelin tire) | Sidewall: 29 | 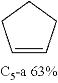 | 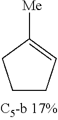 | 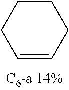 | 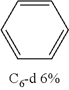 |

| | | | | | |
|---|---|---|---|---|---|
| Tread: 10 | 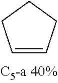 | 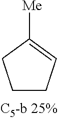 | 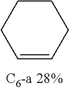 | 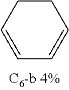 | 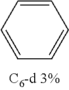 |

| | | | | | |
|---|---|---|---|---|---|
| Example 18 (Pirelli tire) | Sidewall: 18 | 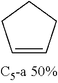 | 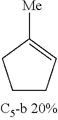 | 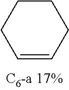 | 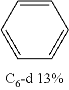 |
| | Tread: 7 | 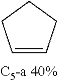 | 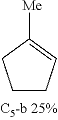 | 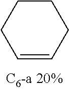 | 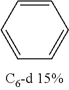 |

Referring to the examples, it was found that (C5-C7)cycloalkene may be effectively obtained from a double bond-containing polymer such as polybutadiene, a copolymer of polybutadiene, and polycycloalkene by the method according to the present invention. Furthermore, it was confirmed that cycloalkene may be obtained from various waste rubbers using the method according to the present invention, and thus, it is expected that chemical recycling of a vulcanized rubber mixture which was difficult to be handled and treated may be realized.

According to an exemplary embodiment of the present invention, substituted or unsubstituted (C5-C7)cycloalkene may be effectively obtained by depolymerization through double bond isomerization and ring-closing metathesis of a double bond-containing polymer such as polybutadiene, a copolymer of polybutadiene, and polycycloalkene, using an isomerization catalyst and a metathesis catalyst. Furthermore, natural rubber, synthetic rubber, vulcanized rubber products of the rubber, rubber powders of the waste rubber products may be depolymerized, using the method according to an exemplary embodiment, to effectively obtain substituted or unsubstituted (C5-C7)cycloalkene. Therefore, the vulcanized rubber or a mixture thereof which was difficult to handle and treat so far may be selectively and chemically recycled to a high value-added cycloalkene compound through the method of the present invention.

Hereinabove, although the present invention has been described by specific exemplary embodiments, they have been provided only for assisting in the entire understanding of the present invention. Therefore, the present invention is not limited to the exemplary embodiments. Various modifications and changes may be made by those skilled in the art to which the present invention pertains from this description.

Therefore, the spirit of the present invention should not be limited to the above-described exemplary embodiments, and the following claims as well as all modifications equal or equivalent to the claims are intended to fall within the scope and spirit of the invention.

What is claimed is:

1. A method for preparing cycloalkene, the method comprising:
depolymerizing a double bond-containing polymer in the presence of an isomerization catalyst and a metathesis catalyst having a N-heterocyclic carbene ligand to prepare (C5-C7)cycloalkene.

2. The method for preparing cycloalkene of claim 1, wherein the double bond-containing polymer is polyalkadiene, polycycloalkene, or a copolymer thereof.

3. The method for preparing cycloalkene of claim 2, wherein the double bond-containing polymer is a polymer derived from (C4-C10)alkadiene or (C5-C10)cycloalkene, or a copolymer including the same.

4. The method for preparing cycloalkene of claim 1, wherein the double bond-containing polymer is rubber.

5. The method for preparing cycloalkene of claim 1, wherein the metathesis catalyst is a ruthenium complex including a N-heterocyclic carbene ligand.

6. The method for preparing cycloalkene of claim 1, wherein the N-heterocyclic carbene ligand is represented by the following Chemical Formula 1: 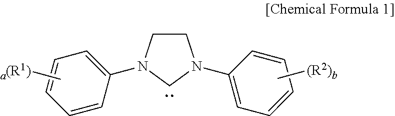
wherein
R¹ and R² are independently of each other (C1-C4)alkyl, and
a and b are independently of each other an integer of 1 to 3.

7. The method for preparing cycloalkene of claim 1, wherein the N-heterocyclic carbene ligand is selected from the following structures:

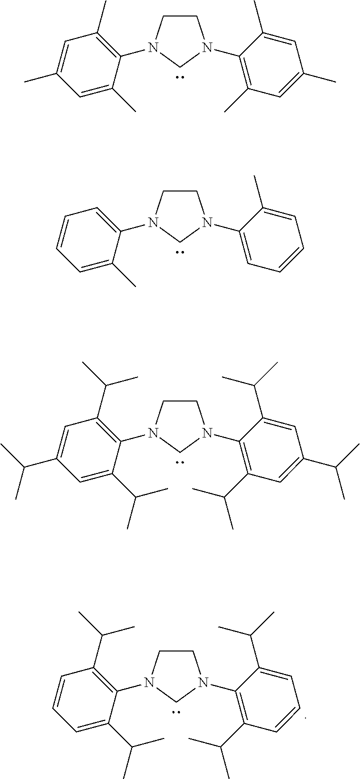

8. The method for preparing cycloalkene of claim 1, wherein the isomerization catalyst includes a phosphine ligand and a central metal.

9. The method for preparing cycloalkene of claim 1, wherein the isomerization catalyst is [Pd(μ-Br)^{t}Bu₃P]₂, RuHCl(CO)(PPh₃)₃, Ru(Me-allyl)₂COD, Ru-MACHO, Pd/C, or Ru/C.

10. The method for preparing cycloalkene of claim 1, wherein the depolymerization reaction is performed at a temperature of 50 to 150° C.

11. The method for preparing cycloalkene of claim 1, wherein the isomerization catalyst and the metathesis catalyst are used at a mole ratio of 1:0.1 to 1.5.

12. The method for preparing cycloalkene of claim 2, wherein the polyalkadiene is selected from polybutadiene, polyisoprene, polypentadiene, and polychlorobutadiene.

13. The method for preparing cycloalkene of claim 2, wherein the polycycloalkene is selected from polycyclooctene, polycyclopentene, and polycyclooctadiene.

14. A method for recycling waste rubber, the method comprising:
grinding waste rubber to obtain waste rubber powder; and depolymerizing the waste rubber powder in the presence of an isomerization catalyst and a metathesis catalyst having a N-heterocyclic carbene ligand to prepare (C5-C7) cycloalkene.
